(19) United States
(12) Patent Application Publication
(10) Pub. No.: US 2008/0016716 A1
(43) Pub. Date: Jan. 24, 2008

(54) Golf balance sandals

(76) Inventor: Adam C. Battaglino, Marlborough, MA (US)

Correspondence Address: MUIRHEAD AND SATURNELLI, LLC, 200 FRIBERG PARKWAY, SUITE 1001, WESTBOROUGH, MA, 01581, US

(21) Appl. No.: 11/488,283

(22) Filed: Jul. 18, 2006

(51) Int. Cl. A43B 3/10; A43B 7/24; A61F 5/14

(52) U.S. Cl. 36/7.8; 36/142; 36/143; 36/144

(57) ABSTRACT

A method and apparatus for a sandal for balance training or muscle strengthening that may be used, for example, in the game of golf. The sandal includes a sole having a toe portion and a heel portion, each of the toe and heel portions including an upper surface and a lower surface. A coupling mechanism is provided that couples the toe portion to the heel portion. At least one of the toe portion and heel portion includes an angled edge adjacent to the lower surface that allows pivoting of the sandal in a forward pivot direction, a backward pivot direction and/or a side cant direction. The coupling mechanism may allow adjustment of a spacing between the toe portion and the heel portion and may include a threaded rod.

### TECHNICAL FIELD

The present application is directed to the field of balance training and muscle strengthening and, more particularly, to a method and apparatus for golf balance sandals.

### BACKGROUND

It is known that special footwear may be used to provide a rocking motion that allows for exercise, muscle strengthening and other therapeutic effects. (See, for example, U.S. Pat. No. 4,206,558 to Bivona; U.S. Pat. No. 5,339,542 to Kim; U.S. Pat. No. 5,507,106 to Fox; U.S. Pat. No. 5,826,351 to Tsuji; and U.S. Pat. No. 6,421,935 to Bartlett, which are all incorporated herein by reference.)

Further, maintaining balance is a desirable skill in many athletic games and events. In particular, in the game of golf, maintaining proper balance during a golf swing may result in a swing having improved accuracy, consistency and/or power.

Accordingly, it is desirable to provide for a system that facilitates balance training and muscle strengthening and that may be used to improve athletic performance, for example, as in the game of golf.

### SUMMARY

According to the present system, a sandal for balance training or muscle strengthening includes a sole having a toe portion and a heel portion, each of the toe and heel portions including an upper surface and a lower surface. A coupling mechanism is provided that couples the toe portion to the heel portion. At least one of the toe portion and heel portion includes an angled edge adjacent to the lower surface. The coupling mechanism may allow adjustment of a spacing between said toe portion and said heel portion and may include a threaded rod. At least one strap may be attached to the sole. The angled edge may be angled in a range of approximately ten degrees to approximately fifty degrees, particularly thirty-five degrees, and may run along a front edge, a back edge or a side edge of the toe portion and/or the heel portion. At least one spring may be disposed on at least one of the toe portion and the heel portion. The spring may be a compression spring or a wave spring and may be so disposed as to generate a cant angle of the toe portion and the heel portion when the spring is in an uncompressed state. The sandal may also include a spike and/or a frictional surface disposed thereon.

According further to the present system, a method of balance training or muscle strengthening during a golf swing includes wearing a pair of sandals during the golf swing, wherein each sandal of the pair of sandals is pivotable in a direction that is at least one of: a forward pivot direction, a backward pivot direction and a side cant direction. Each sandal of said pair of sandals may include a sole having a toe portion and a heel portion, each of the toe and heel portions including an upper surface and a lower surface and a coupling mechanism that couples said toe portion to said heel portion. At least one of the toe portion and the heel portion may include at least one angled edge adjacent to the lower surface. A size of at least one sandal of the pair of sandals may be adjusted by altering a spacing between the toe portion and the heel portion using the coupling mechanism. A front edge of the toe portion may include a first angled edge, and a back edge of the heel portion may include a second angled edge. At least one angled edge may run along a lateral side of the heel portion or the toe portion. At least one spring may be coupled to the sandal that facilitates pivoting of the sandal in at least one of the forward pivot direction, the backward pivot direction and the side cant direction.

The present system provides for a sandal that may ingrain muscle memory and develop balance over an entire golf swing. With continued use, the sandal may strengthen stabilizer muscles throughout the ankles, hips, legs, and/or back. It has been found that limiting the surface area under a foot to the precise area where a golfer's weight should be and remain through the majority of the swing, may force the golfer to focus on staying balanced. If the golfer places too much weight forward or backward he will receive immediate feedback and fall in that direction. Thus, a golfer is forced to maintain balance in order to complete an effective swing. Forcing balance may also steady the body and head which will allow the golfer to make better and more consistent contact with a golf ball. Utilization of the system described herein allows a golfer to gain and foster foundational swing skills in the game of golf through mental awareness of desirable swing dynamics.

### BRIEF DESCRIPTION OF THE DRAWINGS

Embodiments of the system are described with reference to the several figures of the drawings, in which:

FIG. 1 is a schematic illustration of a top-down view of a sandal for facilitating balance training and muscle strengthening according to one embodiment of the system described herein.

FIGS. 2A and 2B are schematic illustrations of side views of the sandal shown in FIG. 1.

FIG. 3 is a schematic illustration of a bottom-up view of the sandal shown in FIGS. 1 and 2.

FIGS. 4A and 4B are schematic illustrations of a front and side view of a sandal with another embodiment of a spring configuration according to the system described herein.

FIGS. 5A and 5B are schematic illustrations of a front and side view of a sandal with another embodiment of a spring configuration according to the system described herein.

FIG. 6 is a schematic illustration of a side view of a sandal with another embodiment of a spring configuration according to the system described herein.

FIG. 7 is a schematic illustration of a front view of a sandal according to another embodiment of the system described herein.

FIGS. 8A-C are schematic illustrations of a bottom view, side view and rear view of a sandal according to another embodiment of the system described herein.

FIG. 9 is a schematic illustration of an applied sandal system showing a sandal strapped to a shoe according to the system described herein.

### DETAILED DESCRIPTION OF VARIOUS EMBODIMENTS

It is to be understood that both the foregoing general description and the following detailed description are exemplary and explanatory only and are not meant to be restrictive of the system described herein. It may be noted that, as used in the specification and the appended claims, the singular forms “a”, “an” and “the” include plural referents unless the context clearly dictates otherwise. References cited herein are hereby incorporated by reference in their entirety.

Referring now to the figures of the drawing, the figures comprise a part of this specification and illustrate exemplary embodiments of the described system. It is to be understood that in some instances various aspects of the system may be shown schematically or may be exaggerated or altered to facilitate an understanding of the system.

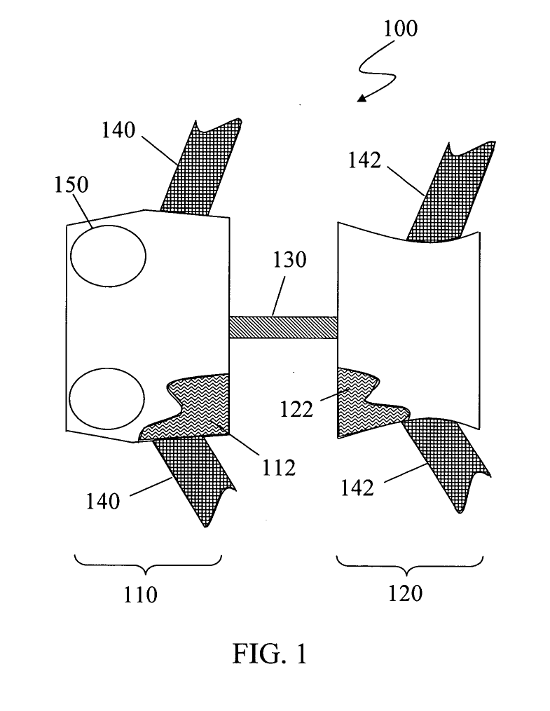

FIG. 1 illustrates a top-down view of a sandal 100 for facilitating balance training and muscle strengthening according to one embodiment of the system described herein. The sandal 100 may include a toe portion 110 and a heel portion 120 coupled together by a mechanism 130 that allows for size adjustment, for example a threaded mechanism, as further described elsewhere herein. The portions 110, 120 may be approximately the same size or may be differently sized. Alternatively, the sandal may include only one integral sole portion.

In various embodiments, the sole of the sandal may be one inch thick, but may range from one-half inch to one and one-half inches in thickness. The core material of the sole may vary and may include 50% wood fiber with 50% polyethylene composite, PVC, nylon, wood, aluminum, fiberglass, rubber, and/or any other appropriate material, such as any hard machineable material. The upper and lower surfaces may have a frictional or no-slip surface 112, 122 either applied or impregnated based on the particular core material used. For example, the surfaces 112, 122 may include a dense foam or rubber surface that partially or fully forms to the contact surface of an applied shoe or foot, thereby facilitating the frictional or no-slip functionality of the interface between the sandal and the applied shoe or foot.

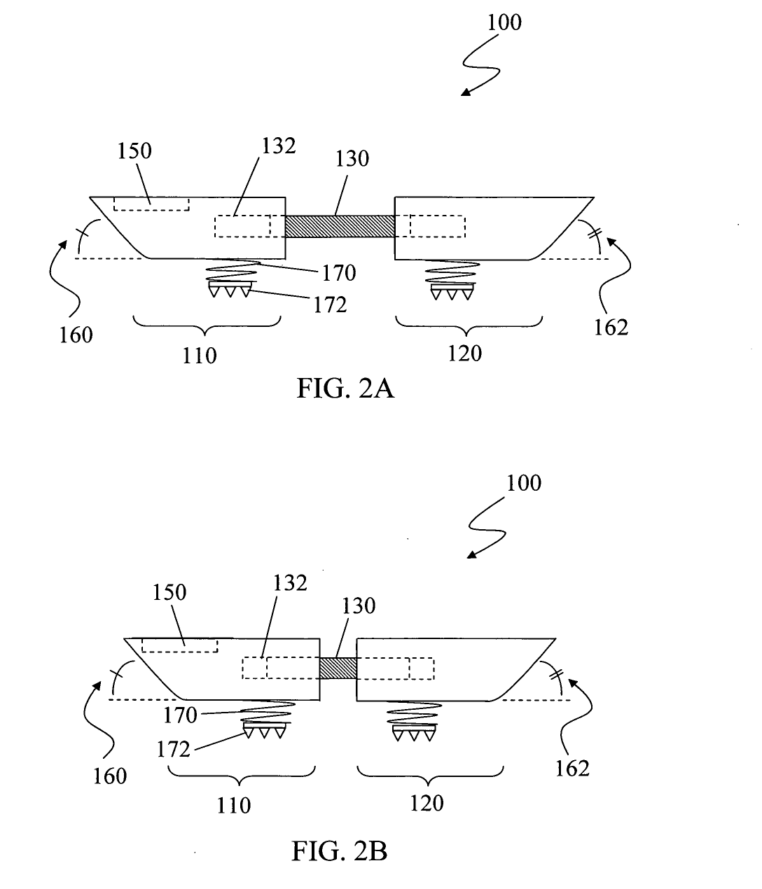

The sole may be split into two portions, approximately the same size, as the toe portion 110 and the heel portion 120. The two portions 110, 120 may be connected by the coupling mechanism 130, such as installing an installed threaded insert or drilling and taping the sole material (shown as an internal cavity 132, see FIGS. 2A and 2B, as further described elsewhere herein) and then using a threaded rod to connect the two portions 110, 120. Other known coupling mechanisms may also be used. The coupling mechanism 130 provides for size adjustment capability of the sandal and allows individual adjustment for each user. The threaded rod used may be a three-eighths inch width rod with sixteen threads per inch. In other embodiments, the threaded rod may be varied from a one-quarter inch rod to a one-half inch rod and include any known thread configuration. The threaded rod's length may vary from two inches to six inches, although a three inch rod and a four and one-half inch rod may cover a wide range of shoe sizes. The sizing of the soles taper and width and length pieces may be varied according to male, female, and child sizing. The threaded rod size and length may also be varied based on male, female or child sizing. The shape of the sole, as shown in the illustrated top-down view of FIG. 1, may conform to the natural flaring out of the soles of most sneakers, golf shoes, hiking shoes, etc., at the toe portion 110 and heel portion 120.

FIGS. 2A and 2B illustrate side views of the sandal 100. As shown, the surface which comes in contact with the shoe may remain flat. The surface which comes in contact with the ground may taper down at front and back pivot angles 160, 162 of approximately thirty-five degrees sloping down towards the toe and heel. In other embodiments, the angles may be in a range of approximately ten degrees to approximately fifty degrees, or a range of approximately fifteen to forty-five degrees, although any appropriate angle may be used. The angle 160 of the toe portion 110 may be the same as or different from the angle 162 of the heel portion 120. It is further contemplated herein that the sandal may have only a front pivot angle (only angle 160 of the toe portion 110) with little or no angle at the heel portion 120 or only a back pivot angle (only angle 162 of the heel portion 120) with little or no angle at the toe portion 110.

A comparison of FIG. 2A with 2B illustrates the size adjustment features of the sandal 100 according to the system described herein. In FIG. 2A, the sandal 100 is shown in an elongated size position in which most of the threaded rod coupling mechanism 130 is used to generate a relatively large spacing between the toe portion 110 and the heel portion 120. In FIG. 2B, the sandal 100 is shown in a compacted size position, with a relatively small spacing between the toe portion 110 and the heel portion 120, in which most of the threaded rod coupling mechanism 130 is contained within the internal cavities 132 of each of the toe portion 110 and the heel portion 120. The size adjustment capability allows for not only adjustment according to the physical size needs of a golfer, but also allows for variable intensity of the balance training experience. That is, generally, the smaller the size setting of the sandal the more difficult it is for a golfer to balance him or herself during the golf swing. Thus, a golfer may initially set the sandal at a relatively larger size position and, thereafter, reduce the spacing between the toe portion 110 and heel portion 120 to increase the intensity of the training and/or exercise experience.

The sandal 100 may include lateral spikes 172 attached to springs 170 that may be seven-eighths inch in diameter by seven-eighths to one and one-quarter inches in height with a wire size that may range between 0.05 and 0.14 inches, although other ranges are possible. In one embodiment, the standard size is 0.08 inches. The player's weight and and/or strength may determine what wire size is comfortable for the player. The springs 170 may be compression or wave springs with equal or varying spring rates, for example as measured in pounds/inch and/or load measured in pounds.

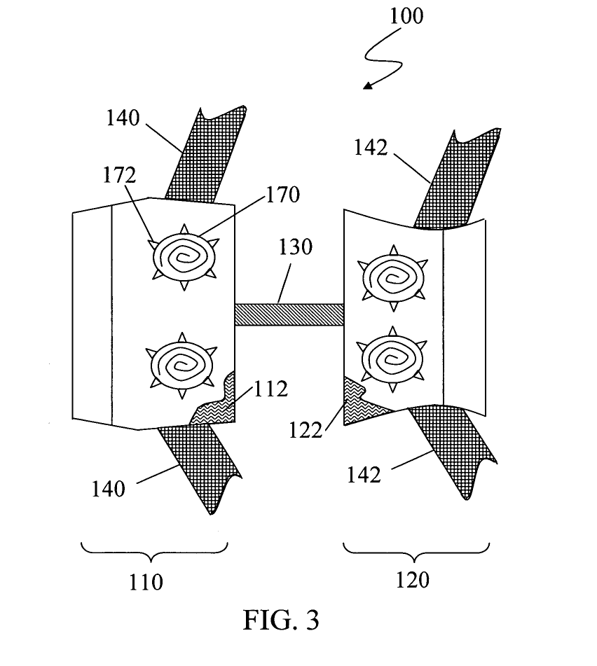

FIG. 3 is a schematic illustration of a bottom-up view of the sandal 100 showing two springs 170 and spikes 172 on each of the toe 110 and heel 120 portions of the sandal 100. For example, the spikes and springs may be attached to the rear foot sandal (right foot for right handed players, left foot for left handed players).

The springs 170 may be useful in connection with training of the golfer. On the back swing, the golfer may feel the sensation of compressing the springs loading the players weight properly on the back foot. On the forward swing, the springs 170 may help to force the golfer to transfer the weight to the front foot and keep it there, while remaining balanced with proper weight distribution throughout the swing. The springs 170 may be press fit into a seven-eighths inch hole drilled one-quarter to three-eighths inches deep. Spacers may be placed in the hole under the spring if more projection is desired.

Alternatively, the spikes 172 may be inserted, without springs, directly onto the flat portion of the sole which comes in contact with the ground. The spikes 172 may be threaded, or inserted through another means, into each bottom portion, front and back halves, of the sandal. It is also contemplated to use springs 170 without spikes.

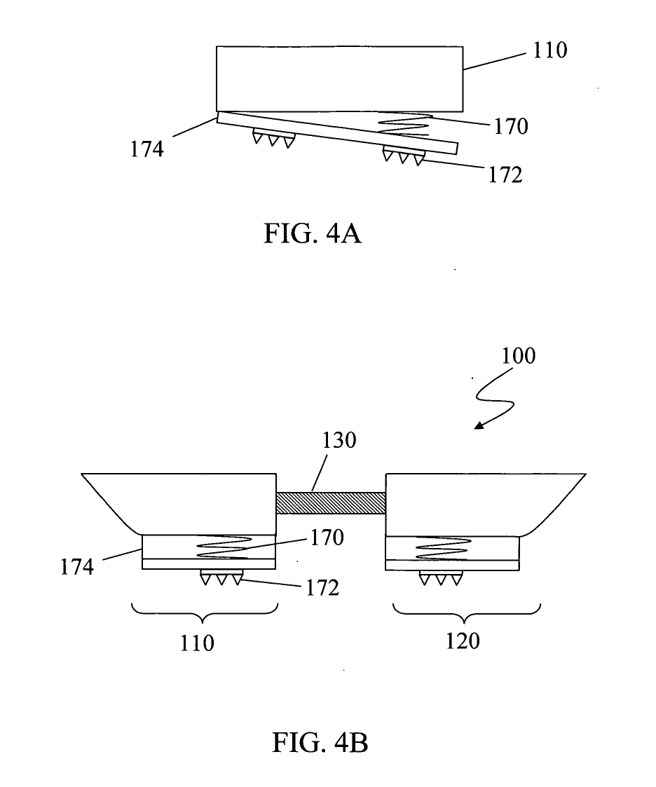

Other configurations of the springs and spikes are possible and contemplated herein. For example, FIGS. 4A and 4B show schematic illustrations of a front view (FIG. 4A) and side view (FIG. 4B) of a sandal with another embodiment of a spring and spike configuration according to the system described herein. As shown in the front view of the sandal in FIG. 4A, the sandal may include a spring 170 on one side of the base of the toe portion 110 (and/or heel portion) and a base support surface 174 attached thereto such that the base support element 174 and spring 170 form a canted bottom surface of the sandal having a cant angle that changes with compression of the spring. Spikes 172 may be attached to the bottom surface of the bottom support element 174. In this manner, further balance training may be provided in a lateral direction as a result of the canted bottom surface of the sandal.

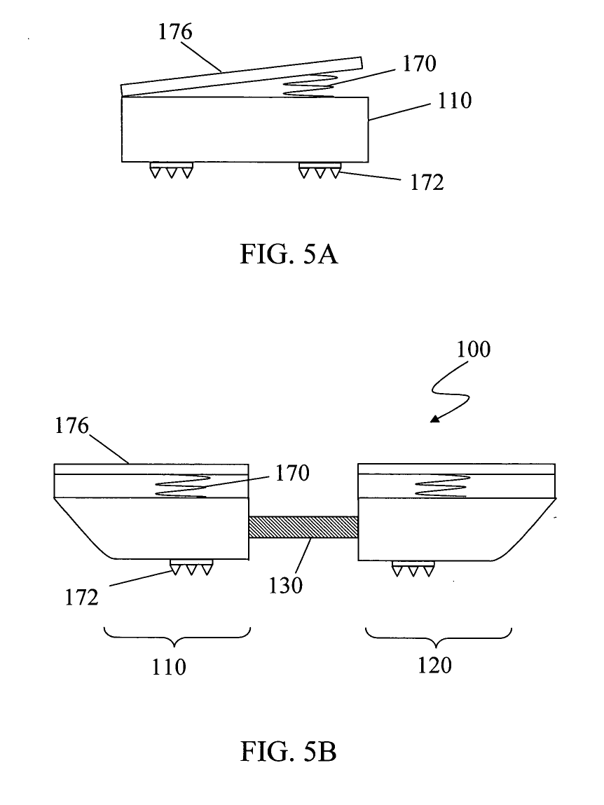

Alternatively, a similar configuration as described above may be incorporated into the top surface of the sandal, that is, the surface in contact with the golfer's shoe. FIG. 5A illustrates a front view and FIG. 5B illustrates a side view of a sandal according to another embodiment of the system described herein showing a spring 170 on one side of the toe portion 110 (and/or heel portion) and a top support surface 176 attached thereto such that the top support element 176 and spring 170 form a canted top surface of the sandal having a cant angle that changes with compression of the spring, for example with maximum cant when the spring is in an uncompressed state. Spikes 172 may be attached to the bottom surface of the sandal. In this manner, further balance training may be provided in a lateral direction as a result of the canted top surface of the sandal.

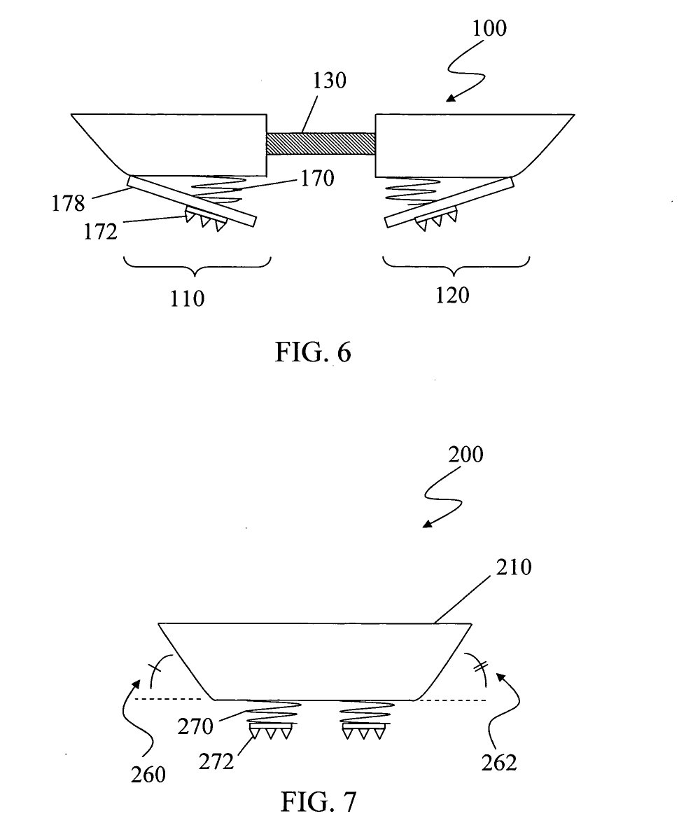

It is further contemplated herein that spring configurations may be provided that facilitate the forward and backward pivot angling of the sandal. For example, FIG. 6 is a side view illustrating a spring configuration for a sandal 100 in which a base support surface 178 is coupled to a spring 170 such that it facilitates angling in the forward and backward pivot directions of the sandal and is configurable for use on the toe portion 110 and/or the heel portion 120 as shown.

FIG. 7 is a schematic illustration of a front view of a sandal 200 according to another embodiment of the system described herein. In the illustrated embodiment, a toe sole portion 210 of the sandal 200 is shown from a front view having cant angles 260 and 262 included on the inside and outside lateral edges of the toe sole portion. In various embodiments, the cant angles 260 and 262 may range from approximately ten degrees to approximately fifty degrees, or a range of approximately fifteen to forty-five degrees, although any appropriate angle may be used. A heel portion of the sandal 200 may have similar cant angles on the inside and outside lateral edges. The sandal also may include springs 270 and spikes 272 configured as described elsewhere herein. As a result, the sandal 200 may provide balance training and muscle strengthening for a golfer balancing in the lateral directions. Consequently, a sandal according to the system described herein may facilitate balancing the in forward and backward directions as a result of the toe and heel pivot angles (160, 162, see FIG. 1) and the lateral directions as a result of the cant angles 260 and 262. It is further contemplated herein that the sandal may have only one lateral edge cut with a cant angle (for example, only cant angle 260 or only cant angle 262).

The top face of the sandal's sole which comes in contact with the shoe's sole may have a recessed area 150 on both front corners (see FIGS. 1 and 2) which may allow for the golf shoe spikes to recess into the sandal 100 thereby accommodating the shoe spike to provide for a comfortable fit of the sandal with standard golf shoes and for protecting the shoe spike from damage.

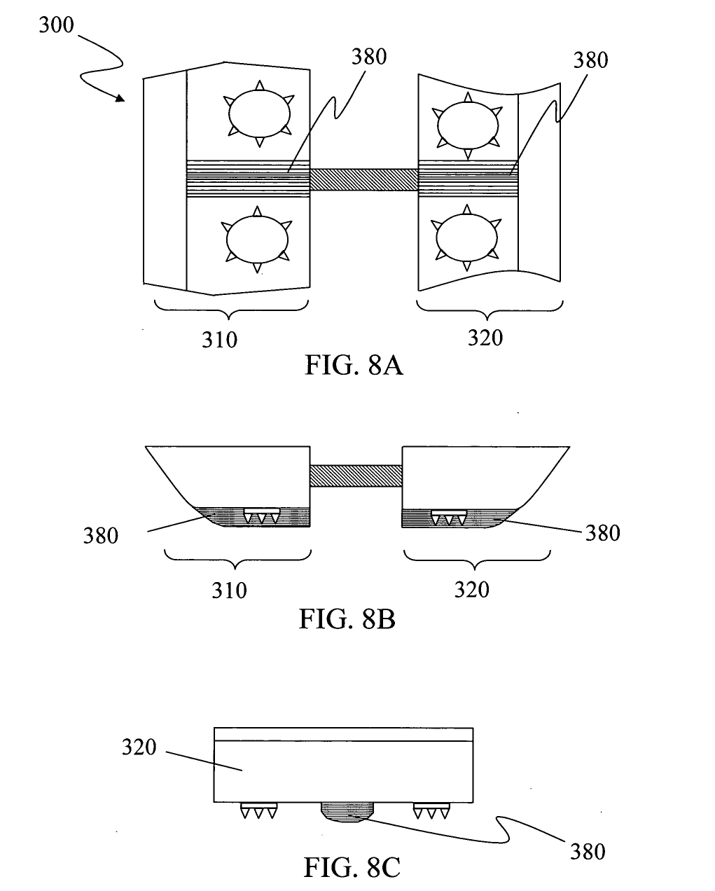

FIGS. 8A-C are schematic illustrations of a bottom view (FIG. 8A), side view (FIG. 8B) and rear view (FIG. 8C) of a sandal 300 according to another embodiment of the system described herein. In the illustrated embodiment, a ridge 380 is shown positioned on the bottom of the sole of the toe 310 and heel 320 portions of the sandal 300. The ridge 380 is shaped to provide an aid for additional medial/lateral balance training and muscle strengthening mechanism of the sandal 300 that facilitates balance in a side-to-side cant direction, for example by having a rounded surface that contacts the ground. In an embodiment herein, the ridge 380 may vary from one-quarter to one-half inches in width and one-quarter to three-quarters inches in height. The outer ends of the ridge 380 may follow the existing taper of the front and rear edges of the toe 310 and heel 320 portions, as shown. The ridge 380 may be used independently or in conjunction with other balance and muscle strengthening mechanisms detailed herein.

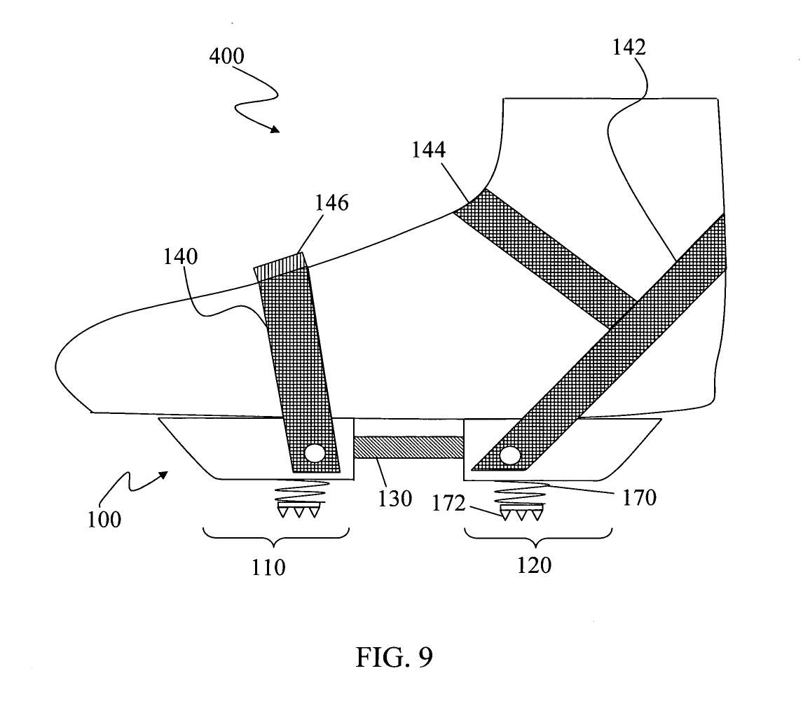

Referring now back to FIG. 1, the sandal may include straps 140, 142 that are made of nylon or leather or any other suitable material which may allow the golfer to firmly attach the sandal to the bottom of the shoe without risk of failure. One strap 140 may be attached to the front sole portion 110 just behind the projection by the ball of the foot. The strap 140 may then wrap around the foot and be firmly attached. Another strap 142 may be attached to the heel sole portion 120 which may go over and around the heel of the shoe and firmly attached. A third strap 144 (see FIG. 9, as described elsewhere herein) may be attached to the rear strap two to three inches up the rear heel strap approximately perpendicular thereto and facing towards the front of the player and secured around the ankle.

The straps described herein may be one or more of the following mechanisms: hook and loop, a buckle system with a ladder lock, quick release cam, metal cam anchor, and/or a sure lock metal tooth buckle system. Other appropriate mechanisms may also be used. The strap mechanisms may provide proper tightening while minimizing risk of unintended release. A buckle system 146 is illustrated in FIG. 9 to provide tightening of the straps.

The size of the sandal 100 may be adjusted for length of the player's foot using the size control features of the coupling mechanism 130, as described elsewhere herein. The sandal may reach the center of the ball of the foot to the front third of the foot's heel. The sizing may be varied based on the balancing requirements of the player.

The following describes an example method for attaching the sandal to a shoe. The toes and ball of the foot may be placed firmly on the ground. The sandal may be placed against the sole of the shoe, and the front portion may be wedged down to the ground between the ground and the ball of the foot. The front toe strap may be attached firmly. Then, the heel strap may be attached firmly. Finally, the ankle strap may be attached firmly to the foot. The process may be repeated for the other foot. The sandals are now on properly and ready for use.

FIG. 9 is a schematic illustration of an applied sandal system 400 showing a foot or shoe on which is strapped a sandal according to the system described herein (for example, sandal 100). Other elements are identified in FIG. 9 as described elsewhere herein in reference to the other figures.

The following embodiment details the use and benefits of the sandals during a golf swing (described for a right handed golfer). First, a golfer (or other user) places their feet a comfortable distance apart with the front foot's toe pointing partway towards the target. With hands and club in front, the golfer may be positioned in the proper balanced posture. For a few practice swings, the golfer may use swings of partial power or intensity to become comfortable with the sandals before commencing the primary swing. On the back swing, the golfer may get the feeling of weight shifting to the back foot. The weight shift may compress the springs in the sandal. Thus, the golfer's body may be balanced but the majority of the golfer's weight is on the back foot and the golfer's body is coiled backwards onto the back leg, thereby storing power for the swing. The golfer's hands and club should be over the right shoulder at the top of the back swing. With the back swing completed, the golfer may then start the forward swing with a hip turn and weight shift from the back to the front foot, while the sandals force the golfer to maintain balance. The club head path is desirably kept inside the target line throughout the swing until impact with the ball. Generally, the head of the club should be kept heading towards, or just to the right of, the target as long as possible. The swing is usually finished with the golfer's hands and club over the left shoulder with the golfer's weight primarily on the left foot.

While swinging, the importance of achieving good balance and weight distribution may be conveyed to the golfer by the feel of the sandals. Over the course of the swing, a proper swing path, from inside to out, may be maintained, during which, while the golfer attempts to maintain his balance, the sandals provide balance and muscle training and build desirable muscle memory. As detailed herein, the golfer may be forced to maintain balance to address the forward and backward pivoting provided by the sandal (see, for example, FIG. 1), and/or the golfer may be forced to maintain balance to address the side canting provided by the sandal (see, for example, FIGS. 7 and 8A-C). As a result of utilization of the present system, the golfer may gain and foster foundational swing skills through mental awareness of desirable swing dynamics.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

What is claimed is:

1. A sandal for balance training or muscle strengthening, comprising:
a sole including a toe portion and a heel portion, each of said toe and heel portions including an upper surface and a lower surface; and
a coupling mechanism that couples said toe portion to said heel portion,
wherein at least one of said toe portion and heel portion includes at least one angled edge adjacent to said lower surface.

2. The sandal of claim 1, wherein said coupling mechanism allows adjustment of a spacing between said toe portion and said heel portion.

3. The sandal of claim 2, wherein said coupling mechanism includes a threaded rod, and wherein said toe portion and said heel portion receive at least a portion of said threaded rod.

4. The sandal of claim 1, further comprising:
at least one strap attached to said sole.

5. The sandal of claim 1, wherein said at least one angled edge is angled in a range of approximately 10 degrees to approximately 50 degrees.

6. The sandal of claim 5, wherein said at least one angled edge is angled at approximately 35 degrees.

7. The sandal of claim 1, wherein said at least one angled edge is at least one of a front edge and a back edge of said toe portion or said heel portion.

8. The sandal of claim 1, wherein a front edge of said toe portion includes a first angled edge and a back edge of said heel portion includes a second angled edge.

9. The sandal of claim 1, wherein said at least one angled edge runs along a lateral side of at least one of said heel portion and said toe portion.

10. The sandal of claim 1, further comprising:
at least one spring disposed on at least one of said toe portion and said heel portion.

11. The sandal of claim 1, wherein said at least one spring is at least one of a compression spring and a wave spring.

12. The sandal of claim 10, wherein said at least one spring is disposed on said sandal as to form a cant angle of at least one of said toe portion and said heel portion when said spring is in a substantially uncompressed state.

13. The sandal of claim 1, further comprising:
at least one spike disposed on at least one of said toe portion and said heel portion.

14. The sandal of claim 1, further comprising:
a frictional surface disposed on at least one of said toe portion and said heel portion.

15. The sandal of claim 1, further comprising:
at least one side cant mechanism that allows pivoting of said sandal in a lateral side direction.

16. A method of balance training or muscle strengthening during a golf swing, comprising:
wearing a pair of sandals during the golf swing, wherein each sandal of said pair of sandals is pivotable in a direction that is at least one of: a forward pivot direction, a backward pivot direction and a side cant direction.

17. The method of claim 16, wherein each sandal of said pair of sandals includes:
a sole including a toe portion and a heel portion, each of said toe and heel portions including an upper surface and a lower surface; and
a coupling mechanism that couples said toe portion to said heel portion, wherein at least one of said toe portion and heel portion includes at least one angled edge adjacent to said lower surface.

18. The method of claim 17, further comprising:
adjusting a size of at least one sandal of said pair of sandals by altering a spacing between said toe portion and said heel portion using said coupling mechanism.

19. The method of claim 17, wherein a front edge of said toe portion includes a first angled edge and a back edge of said heel portion includes a second angled edge.

20. The method of claim 17, wherein said at least one angled edge runs along a lateral side of at least one of said heel portion and said toe portion.

21. The method of claim 16, further comprising:
at least one spring coupled to the sandal that facilitates pivoting of said sandal in at least one of said forward pivot direction, said backward pivot direction and said side cant direction.

22. A sandal for balance training or muscle strengthening, comprising:
at least one sole portion have an upper surface and a lower surface;
a front angled edge adjacent to said lower surface of said at least one sole portion that allows pivoting of said sandal in a forward direction;
a back angled edge adjacent to said lower surface of said at least one sole portion that allows pivoting of said sandal in a backward direction; and
a side cant mechanism that allows pivoting of said sandal in a lateral side direction.

23. The sandal of claim 22, wherein said side cant mechanism includes at least one side angled edge.

24. The sandal of claim 22, wherein said side cant mechanism includes at least one spring disposed on at least one of said upper surface and said lower surface that forms a side cant angle of said sandal when said spring is in a substantially uncompressed state.

25. The sandal of claim 22, wherein said at least one sole portion includes a toe portion and a heel portion and wherein said front angled edge is disposed on said toe portion and said back angled edge is disposed on said heel portion.

26. The sandal of claim 25, further comprising:
a coupling mechanism that couples said toe portion and said heel portion and that allows for adjustment of a spacing between said toe portion and said heel portion.
